(19) United States
(12) Patent Application Publication
(10) Pub. No.: US 2015/0302581 A1
(43) Pub. Date: Oct. 22, 2015

(54) METHOD AND DEVICE FOR COSMETIC DENTAL ANALYSIS

(71) Applicant: Carsten DURSTELER, Berlin (DE)

(72) Inventor: Carsten Dursteler, Berlin (DE)

(21) Appl. No.: 14/439,713

(22) PCT Filed: Nov. 4, 2013
(86) PCT No.: PCT/EP2013/072929

§ 371 Date: Apr. 30, 2015

(30) Foreign Application Priority Data
Nov. 2, 2012 (DE) 102012110491.3

(51) Int. Cl. G06T 7/00; A61C 19/04; G06F 3/0484; A61B 5/00; G06T 11/60; G06F 3/0488

(52) U.S. Cl. CPC G06T 7/0012; G06T 11/60; G06F 3/0488; G06F 3/04847; A61B 5/0077; A61B 5/0088; A61C 19/04; A61B 5/0013; A61B 5/742; A61B 5/7475; G06T 2200/24; G06T 2207/20104; G06T 2207/30036

(57) ABSTRACT

The invention relates to a method and a device for cosmetic dental analysis. According to the invention, orthodontic parameters of a person are acquired, analyzed, and represented by means of a camera, a computer and a screen.

### PRIOR ART

In the field of cosmetic dental analysis and dental consultation, methods or devices are described, which capture the relevant parameters for dentistry work in a mechanical manner. Thus, in the prior art, impressions of the jaws and teeth are prepared as the foundation for dentistry work. The technician prepares plaster models with the aid of these impressions, which reproduce the state in the mouth of the affected person. In addition, the location relationship of the jaws in relation to one another is captured in an articulator by simulating the mandibular joint movement. Such work using models is established in the field of dentistry and in the prior art.

The use of these models has the disadvantage that the entirety of the cosmetically relevant parameters are not captured in the case of the affected persons, whose teeth are to be subjected to cosmetic adaptations. Only teeth and jaws are represented, in a manner artificially modeled by impressions and plaster models. In addition, these methods are relatively imprecise, since the persons often freeze up during the preparation of the impression and therefore the natural situation is not reproduced.

In addition, compositions and methods for whitening teeth are known from the prior art. Such compositions and methods are described in DE 19581846 T1 or EP 2376018 A1.

Mechanical devices are also described in the prior art, by means of which cosmetically relevant parameters are captured, by manually recording the laugh line and lip line or canine guidance as lines on a Plexiglas plate. This type of capturing cosmetically relevant parameters also makes use of the preparation of a model, to carry out the dentistry work later. This method of model preparation is therefore also linked to inaccuracies and deviations from the original situation in the mouth of the affected person.

To prevent such inaccuracies, complex and costly laser optic methods using micro-endoscope cameras have been described, which prevents flexible and practical application, however. In addition, such methods have the disadvantage that sometimes substantial unpleasantness results for the affected person due to the production of the model.

In relation to the color selection in the case of teeth, an establishment is performed in the prior art by means of comparison tables, as to which is the matching color in the cosmetic tooth adaptation. Merely comparing colors is linked to disadvantages, because it only reflects the subjective impression of the observer. Simple, uncomplicated, and flexible methods by means of image recognition and image processing are already known in the field of hair color consultation, which work with a static, digital portrait picture and wherein optionally a desired hairstyle color is input and displayed on a display screen. Methods using moving images are also used for hair color consultation (EP1527713B1).

A method for effective planning and visualization and also optimization of dental reproductions is disclosed in document WO2010/105628. In this case, firstly a digital 3-D model of the teeth to be prepared is provided. Subsequently, a CAD model is designed, which is based on the 3-D model. In this document, the advantages are described, which are achieved in that the 3-D model of the teeth to be reconstructed can be shown to the patients before the treatment. However, this already relates to the finished model in this case, which displays the state after the treatment. The document does not describe an extensive analysis, which is necessary, however, to be able to carry out a comprehensive cosmetic dental consultation.

In this document, it is furthermore described that multiple items of biometric information can be used for the optimization process. The cited method has the disadvantage, however, that complete modeling of the reproduction process or the processing process always takes place here. The presence or work of a dentist, oral surgeon, or orthodontist is thus necessary. This type of representation, specifically as a CAD-3-D model, is still costly and relatively time-consuming for the mentioned reasons.

The object of the present invention is to provide a method and a device for a cosmetic tooth analysis, which does not have the disadvantages and deficiencies of the prior art.

### DESCRIPTION

The object is achieved by the independent claims. Advantageous embodiments result from the dependent claims.

In a first preferred embodiment, the invention relates to a method for a cosmetic dental analysis, wherein cosmetic parameters of a person are captured, analyzed, and displayed by means of a camera, a computer, and a display screen, characterized in that the method comprises the following steps:
a. recording an image of the face of the person by means of a camera,
b. transmitting the image to the computer,
c. image editing and image processing,
d. marking the cosmetically relevant parameters on the image,
e. carrying out an analysis on the basis of the position markings by way of the computer,
f. displaying the result in real time on the display screen.

This preferably relates to a computer-implemented method. This preferably relates above all to steps b) and c) and to steps d) and c). Due to the use of a computer-implemented method, the dental analysis to be performed is simplified and accelerated in manifold ways.

It is preferable if, by way of the method of the present invention, an image region is selected after transmitting the image to the computer. In this case, it is also possible that multiple images are recorded and multiple images are transmitted. Different image regions of either one image or of multiple images can thus also be selected.

The image region or the image regions are preferably selected so that the same mouth region is visible. Furthermore, it is preferable if either exclusively or additionally an image region is selected, on which both the mouth region and parts of the nose are visible. This is advantageous since thus an overall dental analysis can be performed. By also recording the lower part of the nose in the image region, the dental consultation and dental analysis can additionally be performed on the basis of features of the respective face. Better results can thus be achieved than if individual teeth are only considered per se.

It is preferable for the camera to take portrait and close-up pictures, and for the images to be displayed by at least one display screen as a mirror image.

It is particularly preferable for the first image recording to be a portrait image and a second image recording to be a close-up picture of the mouth region and/or mouth and nose region. This embodiment is preferable above all if the resolution of the camera is not particularly high. By recording a close-up region, the quality can therefore be improved in relation to a digitally enlarged image detail.

Furthermore, it is preferable if the image is transferred from an existing file on the computer. The task of the computer is to modify the image so that the result of a cosmetic dental analysis can be displayed. For this purpose, the mouth and tooth region of the person must be able to be recognized on the image, and by inputting the cosmetically relevant parameters during the cosmetic dental analysis, the computer, comprising the unit for image editing and image processing, recognizes the cosmetic adaptations on the teeth of the affected person. In this case, the relevant points or characteristics are preferably marked on the display screen. These include in particular the position of the center line, the laugh line, the color of the teeth, the width of the smile, the tooth axes, the length and width of the individual teeth, the golden ratio, the interdental triangles, the red/white aesthetics, and the gum papillae.

It is preferable for initial parameters to be communicated to the computer for characterizing an image region, preferably for characterizing a tooth and mouth region.

It is preferable for the device to continuously process manually-input initial parameters of an image region for initialization, wherein the tooth and mouth region is marked as at least one initial parameter.

Initial parameters can also preferably be referred to as starting parameters. The starting parameters characterize the actual state and comprise all important characteristic parameters for an aesthetic dental analysis and dental evaluation. These parameters can be captured and displayed in a simple, rapid, and particularly comprehensive manner, which is illustrative to the patient, for the first time by the invention.

It is preferable if initial parameters for characterizing an image region, preferably for characterizing the tooth and mouth region, are communicated to the computer. The initial parameters are particularly ascertained during the cosmetic dental analysis. In particular before a simulation of a cosmetic dental correction, the initial parameters are communicated to the computer (initialization). For this purpose, the device continuously processes manually-input initial parameters of an image region, wherein, for example, a mouth and/or tooth region is marked as at least one initial parameter. In particular, the teeth to be corrected can be marked or, for example, other parameters identified during the dental analysis can also be marked. It is preferable if all cosmetically relevant parameters can be input and marked. In particular, the cosmetically relevant parameters are particularly preferably center line, laugh line, color, width of the smile, tooth axes, golden ratio, length-width ratio, interdental triangles, red-white ratio, and/or gum papillae.

As further combinations of initial parameters, it can be predefined which image regions are a tooth region and a gum region, and whereby these are characterized (color, morphology, shape), which (natural) starting situation prevails, and which cosmetic dental adaptation is desired. This can be performed by selecting or marking image regions or buttons of one or more operating menus on the display screen or touch screen. In particular, the cosmetically relevant parameters can be input or marked. The input device is required for this purpose can be provided, for example, as a mouse or the like (trackball, touchpad, etc.), as a keyboard, and/or as at least one touch screen. The initialization phase can be necessary, inter alia, to communicate to the computer which image regions of the mouth and tooth region represent the cosmetically adapted tooth region, which is to be separated from the remaining image and reworked according to cosmetic criteria. This can be performed, for example, in that the user marks one or more points or one or more regions at least in one of the image regions of the teeth, the gums, or the background. In addition, it is possible that teeth are marked, which are explicitly not to be adapted. It is particularly preferable for the computer to be capable after a single input of recognizing these image regions and individual positions and immediately displaying them.

To make it easier for the computer to recognize the affected teeth and to keep the error susceptibility of the dental consultation system low, it is advantageous to record an image of a person in front of a homogeneous, monochrome background with defined illumination if possible.

It is particularly preferable in this case for the selection of the image region to be performed by means of image editing and image processing. A close-up picture of the mouth region having visible upper row of teeth is particularly preferably selected on the display screen.

The display screen preferably comprises a touch screen. Above all, the simple operability and flexibility of the handling are advantageous in this case.

It is preferable for the cosmetically relevant parameters to be input into the computer optionally by at least one display screen, preferably a touch screen, by an input keyboard, or by a mouse.

It is preferable for the cosmetically relevant parameters to be selected from the group center line, laugh line, color, width of the smile, tooth axes, golden ratio, length-width ratio, interdental triangles, red/white ratio, and/or gum papillae.

It is preferable if by way of the method of the present invention, for a cosmetic dental analysis, it is visualized in particular at which positions in the tooth and mouth region dentistry work is to be performed. It is thus possible to provide a comprehensive dental consultation. This dental consultation relates not only to individual aspects, but rather the overall aesthetics of the entire dental region. For this purpose, a dental analysis is carried out and the cosmetically relevant parameters are input into the computer and/or displayable in the computer.

The result of the cosmetic analysis of the invention is preferably displayed on the display screen before the actual dentistry work or the cosmetic adaptations. In particular in the field of cosmetic dental analysis and dental consultation, a visualization of the relevant parameters before the actual cosmetic adaptations of the teeth is significant when consulting with the affected person. It is thus surprisingly possible using the present invention to overcome an inhibition threshold in the case of the person on whom the cosmetic adaptations are to be carried out. Using most methods in the prior art, the affected person does not know beforehand what the result of the cosmetic adaptation of the teeth will be and how the teeth will appear thereafter. A further disadvantage in the prior art is that the methods therein already cause a high consumption of time and costs, before the actual work is performed on the teeth. This means that the patient or the person to be treated must already invest a large amount of time and money, even if he is unsatisfactory with the planned result and decides against the treatment for this reason.

In addition, a comprehensive consultation can be performed for the first time by the method according to the invention and it is possible to visualize all aspects and thus also explain them. The measures to be performed can therefore be discussed substantially more extensively and specifically, which contributes to increasing satisfaction many times over.

The method according to the invention is particularly simple and does not require a large amount of time. Therefore, a person can be shown, in a simple and rapid manner, at which points processing of the teeth or the mouth region could be performed. The inhibition threshold for such a nonbinding dental analysis and consultation is therefore low. If the person decides against the treatment, at this point in time, high costs have not yet occurred. A large amount of time has also not yet been invested on the part of the consultant.

It is possible by way of the method according to the invention for the first time to display the cosmetically relevant points in the tooth and mouth region in a simple and illustrative manner. Many persons do not know which parameters and above all which interactions of individual parameters have effects on the overall aesthetic impression of the tooth and mouth region. This can be visualized and explained in a particularly simple manner by way of the invention.

It is preferable if the method assists the cosmetic consultant or technician in his consultation activity and using the present invention, for example, a two-dimensional image, in particular a digital photo of a person, is recorded and displayed on a display screen, wherein the cosmetically relevant parameters can be visualized independently of the model. The cosmetically relevant parameters in the tooth and mouth region are captured on the basis of the cosmetic dental analysis. Markings are positioned on a touch screen for this purpose, for example. The cosmetically relevant parameters are displayed on a display screen in particular by means of a computer, which comprises in particular a unit for image editing and image processing.

It is preferable if, because no mechanical models are required, the result is displayed without a time delay, the result can be processed and the person, on whose teeth cosmetic adaptations of a technical nature are to be performed, sees immediately at which positions the adaptations will occur. The affected person therefore immediately receives a conception of the result to be expected, of how the corrections will appear after the cosmetic adaptation is carried out on the teeth. It is preferable if not only an analysis of the actual state is performed using the method of the invention, but rather a simulation of the possible result of the change can also be performed. The representations on the display screen do not provide a distorted, modeled image in this case, but rather reproduce the simulated cosmetic tooth adaptation naturally. One reason why the results of conventional dental consultation systems have an unnatural appearance in the prior art is that the analysis and consultation is not carried out on the basis of the visual representation of the actual teeth, but rather by means of a replica and models of teeth. The actual conditions in the mouth and tooth region of the affected person to be advised are thus reproduced in a manner which is not faithful to nature and is only approximate. In addition, the teeth cannot be judged in the interaction with the overall face. A model cannot replace a living, natural image, as the person experiences it, for example, when he observes his teeth in the mirror after the cosmetic adaptation.

However, this only relates to a particularly preferred embodiment. It is also preferable for no visualization of the reworking to be performed to be displayed. The invention would relate in this case only to displaying the actual state on the basis of specific parameters.

In contrast to existing devices and systems for dental consultation, the method of the present invention is based, on the one hand, on an image of the actual teeth and not on a model, and, on the other hand, the starting points for a cosmetic tooth adaptation, which are identified in the cosmetic dental analysis, can be displayed and optionally simulated on the image of the natural teeth of the affected person. The image is processed by the method in real time or nearly in real time (i.e., with a delay which is hardly perceptible to the user) and reproduced on a display screen, so that the impression of using a mirror results. The cosmetic dental analysis and the simulation of the result of a cosmetic dental adaptation can preferably be performed independently of one another. Thus, for example, using the method and the device of the present invention, firstly the cosmetic dental analysis can be carried out and the cosmetically relevant parameters can be captured and stored in the computer. These parameters can be retrieved at any time and used in a cosmetic dental consultation, which is linked in particular to a simulation of the cosmetic dental adaptations. Each cosmetic adaptation in the mouth and tooth region can be displayed in real time or nearly in real time on the display screen in particular.

The invention preferably simulates the observation of a non-reversed image or a separate mirror image. For this purpose, the current image of the affected person is recorded using a camera, processed by a computer, and displayed on a display screen. The display screen becomes the mirror by way of this dynamic representation in real-time or nearly in real time.

The following cosmetic parameters are preferably analyzed:

It is preferable for the position of the center line to be displayed. This is used above all to check a possible deviation in relation to the face center. The marking of the laugh line is to indicate whether the curve of the incisal edges of maxillary anterior teeth harmonizes with the curve of the lower lip when smiling. The line is preferably placed along the incisal edges of the upper row of teeth in this case. It can thus be visualized whether this line extends close to the lower lip and whether the shape of the lower lip corresponds.

The judgment of the tooth color or tooth discolorations is preferably performed for all teeth. However, it can also be advisable in some cases to judge individual teeth separately. The front teeth and incisors are particularly important in this case, since they are of greater significance for the overall aesthetic impression.

During the analysis of the width of the smile, the size and position of the buccal corridor is evaluated.

By way of the marking of the tooth axes, the tooth position and the tooth inclination of individual teeth can be evaluated and analyzed.

The so-called golden ratio describes a harmonic width ratio of the front teeth to one another. In this case, the ratio of 1.6/1.0/0.6 is particularly preferred.

The marking of the length and width of the individual teeth is used for judging the tooth shape, a length/width ratio of 70 to 80% is ideal.

It can be analyzed by the marking of the interdental triangles whether the row of teeth has a loosened or pushed together appearance. In this case, the triangles should become larger proceeding from the center of the face.

By marking the red/white aesthetics, the gum curve can be analyzed from the middle incisor to the canine on both sides. In this case, the lateral incisor should be somewhat shorter than the tangent between the canine incisor and the canine incisor.

It can be judged by the marking of the gum papillae whether they are filled out well. By way of the marking of these relevant parameters for dental aesthetics, it can be shown to the respective person at which points improvements could be performed. Since the respective persons are usually already very accustomed to the appearance and the formation of their own teeth, a consciousness of possible changes can be provided by this dental analysis.

The displayed mouth and tooth region of the affected person can also be changed on the basis of the specifications of the cosmetic consultant or technician, and in this manner a naturally appearing simulation result arises. Since in particular the affected teeth can be displayed in adapted form, for example, in changed shape, color, or size, the effect of unnaturally appearing models is dispensed with. The person can see the result of the simulation comparably to a virtual mirror and can judge himself whether the selected cosmetic adaptations to the teeth correspond to the expectations. If not, other cosmetic corrections can be simulated immediately.

The sequence of the method for a cosmetic dental analysis and dental consultation in the meaning of the present invention is, for example, in that by means of a camera, a computer, and at least one display screen or one touch screen, a selection and display of the desired cosmetic adaptations on the teeth of an affected person in particular are displayed on the display screen and/or touch screen. The image recorded using the camera is transmitted to the computer. Image editing and image processing is carried out therein for each image by means of a unit. A close-up picture of the mouth region having visible upper row of teeth is preferably selected on the display screen by means of image editing and image processing. Furthermore, it is preferable if the computer identifies a mouth and tooth region of the affected person by segmenting, which means recognition and separation of relevant image regions. In addition, it is preferable for image improvement to be carried out automatically using the image processing unit. In this case, color, brightness, and/or contrast of the image are preferably optimized. If an image does not have the required image quality for the subsequent dental analysis, i.e., is too fuzzy, for example, the computer-implemented method preferably provides a corresponding instruction, so that the processor is requested to record the image again. It is therefore ensured that a high quality image is always available for the dental analysis and the quality of the analysis itself is thus also improved.

Furthermore, properties of the teeth, in particular morphology, color, shape, and size, for example, are to be input into the computer. The image is preferably thus changed, so that at least the parameters are marked or emphasized. The changed image is displayed on at least one display screen or touch screen, preferably in real time. Real time in this context means a time delay which is not perceptible or is hardly perceptible by a person between the image recording and/or image processing and the display on the display screen or touch screen.

The mouth and tooth region to be cosmetically adapted is marked and analyzed by means of the dental analysis. Therefore, deviations from the ideal image can be illustrated. Optionally, the desired tooth adaptations are additionally made visible on the display screen and the desired cosmetic tooth adaptation is simulated in the marked region of the affected teeth using the unit of the computer in a simple reworking method. For this purpose, a representative partial region of the marked teeth is manually or automatically selected for the analysis and the analysis is then started, whereby the unit only considers the relevant tooth region for the continuous image processing. This marking is based on the consideration that the teeth have a coherent area of apparent and similar pixel color values, which may be clearly differentiated from the region of the gums. The tooth region can optionally be automatically or manually marked as a coherent texture and/or coherent morphological properties, to clearly differentiate the remaining image region. Depending on the field of application, for example, in a dental laboratory, in addition to the person on which the cosmetic tooth adaptation is to be carried out after the cosmetic dental analysis and dental consultation, and the cosmetic consultant, a technician can also be present. In this case, it is advisable if the technician has a separate, second display screen or touch screen available for the program operation and startup. On this second display screen or touch screen, in addition to the image or image detail of the mouth and tooth region, further items of information which are important for operation can be displayed. These relate, for example, to the possible cosmetically adaptable parameters, for example, color, shape, size, and surface properties of the teeth, so that on the first display screen or touch screen, only the result of the cosmetic dental analysis and dental consultation and/or the simulation result for the cosmetic dental consultation have to be made visible for the person on the first display screen or touch screen. The touch screen has the advantage that further input devices are superfluous, since appropriate operating menus or buttons can be activated directly by tapping using a finger or pen on the touch screen surface.

In one preferred embodiment, the image of the person whose teeth are to be subjected to a cosmetic dental analysis and dental consultation is reflected by the computer about a vertical axis and displayed on the display screen as a mirror image. The impression is thus created of looking at oneself in a mirror, which meets everyday expectations and promotes the unforced interaction with the device for dental consultation. To minimize a parallax between the camera and the display screen or touch screen, the camera should be positioned close to the upper edge of the display screen. In the ideal case, the optical axis of the image is coincident with the optical axis of the display screen or touch screen, to completely prevent parallax. To perfect the effect of a mirror, the imaging scale of the person and the display screen or touch screen should be approximately 1:1.

It is preferable if the representation of the color of the teeth to be cosmetically adapted is variable continuously and in a cycle according to a color wheel surface spectrum during the cosmetic dental analysis, wherein a start or stop of this running color variation is provided. A targeted input or selection of the matching color of the teeth to be corrected from a color palette is thus omitted, for example. The goal of the color selection is not to be able to determine a difference from the teeth which are not to be corrected. By way of the continuously running cycle, at the moment when the affected person or the cosmetic consultant believes they have found the color tone matching with the teeth which are not to be corrected, the cycle of the running color variation can simply be changed and the defined color can be input into the computer. The cycle of the running color variation is freely selectable in its repetition frequency. This running cycle of the tooth change, in particular the tooth color, is particularly well suitable for an advertising demonstration for a cosmetic dental analysis and dental consultation in a dental laboratory, for example, wherein a corresponding video can optionally be displayed on the display screen for the advertisement.

The input of additional items of information, in addition to the input of a recorded image, which can be uploaded from a file, for example, can be performed, for example, by the cosmetic consultant or technician into the computer. These additional items of information comprise in particular the cosmetically relevant parameters of the affected person, but also general or personal data of the affected person, for which the method of the present invention is to be carried out. The representation of the desired tooth correction on the basis of reference teeth or reference parameters can also be input into the computer or selected from a file, for example, to display differences from the natural teeth which are not to be corrected and the simulation result. Targeted work instructions or marking can also be input into the image of the mouth and tooth region to be corrected by dentistry, wherein the input sequence is arbitrary. These data enter an initialization unit, in which the method is started, and a segmenting unit, in which recognition and separation of relevant image regions is performed. After a transformation of the teeth in a transformation unit on the basis of the input cosmetically relevant parameters after completed cosmetic dental analysis and/or dental consultation, a corresponding image display to the client is performed with the adapted teeth on a display screen or touch screen. However, it can also be provided that not all cosmetically relevant parameters are sufficient for initialization. For example, it can be defined by means of input on the computer which parameters or specifications are automatically captured and processed or can already have been uploaded from an existing file. In particular, it can be established which cosmetically relevant parameters are to be considered. In this case, the term cosmetically relevant parameters also comprises dentistry relevant parameters, for example.

Since only a partial aspect of the real image is modified, in particular there are no discrepancies between real and virtual image elements, for example, angle and position inaccuracies, differing illumination of the various image elements, scaling errors, etc. There are also no concealment problems, since it does not have to be calculated which parts of the virtual image element are concealed by the real image. Since the real image is only changed in the defined and cosmetically relevant parameters, the teeth of a person automatically behave physically correctly, which causes a natural effect of the person upon the display on the display screen or touch screen.

The invention relates to a device for a cosmetic dental analysis and dental consultation comprising a camera for recording at least one image of a person, a computer, to which the image is transmittable, preferably in real time, a unit for carrying out image editing and image processing for the image, wherein a tooth and mouth region of the person can be analyzed using the computer by marking the cosmetically relevant parameters on the display screen by means of the position markings and the result can be displayed on at least one display screen, preferably in real time or nearly in real time.

It is preferable if the device comprises a touch screen, in particular a touch screen for communicating initial parameters to the computer for characterizing an image region, which is a tooth and mouth region in particular.

The invention furthermore relates to a dental laboratory, in particular having a device as defined in the present invention.

### EXAMPLE

The invention will now be explained on the basis of an exemplary embodiment. The examples are preferred embodiment variants, which do not restrict the invention.

It is preferable for the cosmetic dental analysis to comprise the capture of personal master data in particular. In this case, for example, the instruction is to be input into the computer by means of a touch screen as to whether a point score evaluation is to be performed. If a point score evaluation is to be performed, the point evaluation is overlaid during the individual steps. If no point score evaluation is to be performed, the point evaluation is blanked out during the individual steps.

The method according to the invention comprises in particular recording an image. It is necessary in this case, for example, that the eyes of the affected person, for which the cosmetic dental analysis and dental consultation are to be performed, are open during the image recording, that the person smiles in particular, and that the upper row of teeth is visible. Following the recording of the image, preferably a portrait image, the eye line (bipupillary line) is to be checked in particular, and specifically in relation to the curve of the upper row of teeth (or occlusal plane). The lines preferably extend in parallel. The image recording is performed, for example, using a camera installed in a touch screen. The image recording is preferably performed using a tablet computer.

During the performance of a cosmetic dental analysis and dental consultation, the images recorded using a camera are preferably aligned horizontally and the images are in particular compulsory pictures in a suitable input mask, for example, on the display screen and/or touch screen. An image detail, preferably of the mouth and tooth region, can be selected and stored via the selection menu on the touch screen of the tablet computer in particular. Instead of recording a portrait image using the camera, an image already stored on the computer can also be loaded or selected.

In the further method, for example, after recording a portrait image, a second image is recorded, which is in particular a close-up picture of the mouth. The upper row of teeth is to be visible in this case. This image is used in particular for judging the rows of teeth for the lip image. As the third image, for example, a close-up picture is recorded of the mouth and a part of the nose, wherein in particular the transition to the gingiva is to be visible, optionally with the aid of a cheek retractor. This third image is used in particular for the detailed evaluation of the teeth and of the red-white ratio.

During the cosmetic dental analysis, all cosmetically relevant parameters are preferably captured. This is carried out in a 10-point checklist, for example. Thus, for example, during point (1), the position of the center line is defined. The image to be judged is tapped once on the touch screen for this purpose in particular. A vertical line appears. This vertical line can be shifted using a circle which is visible on the touch screen. A small circle displayed on the touch screen is the axis of rotation and a displayed semicircle having two arrows pivots the axis of rotation. The positioning of the center line as a dentistry relevant parameter is used in particular for checking a possible deviation from the center of the face.

During point (2) of the cosmetic dental analysis, the laugh line is preferably defined. The laugh line is defined via four markers. The line is placed along the incisal edges of the upper row of teeth. The curve of the incisal edges of the maxillary anterior teeth is to harmonize with the curve of the lower lip. Deviations are made visible by the visualization. To define the laugh line, the image displayed on the touch screen is tapped once on the left at the incisal edges and then once on the right at the incisal edges. A yellow line appears. The two outer tabs displayed on the display screen move the endpoints of the yellow line, which represents the laugh line. The line curve of the laugh line is moved using the inner two tabs.

During point (3) of the cosmetic dental analysis, the tooth color or tooth discolorations are preferably analyzed.

As point (4) of the cosmetic dental analysis, the width of the smile is preferably defined. For the definition under point (4) of the cosmetic analysis, the image is tapped on the left and right in each case in the region of the corner of the mouth. Two yellow brackets now appear in each case. A larger circle moves the pair of brackets, a smaller circle moves the spacing of the brackets. This determination comprises the evaluation of the buccal corridor, which are displayed, for example, as dark triangles on the display screen. The displayed brackets mark the respective dark region if present.

As point (5) of the cosmetic dental analysis for defining the cosmetically relevant parameters, the tooth axes are preferably defined. For this purpose, the image displayed on the touch screen is tapped in each case for each desired tooth axis, for example, six times. A yellow line appears in each case. A large circle on the touch screen moves the yellow line. A small circle represents the axis of rotation. With the aid of the large circle, the axis of rotation is positioned in the tooth center point. The axis inclination is set using the semicircle. An evaluation of the tooth position and inclination is performed using the evaluation under point (5) during the definition of the tooth axes. Possible deviations are defined and visualized by positioning or inclining the axis.

As point (6) of the cosmetic dental analysis, the golden ratio is preferably defined. The golden ratio describes a harmonious width ratio of the maxillary anterior teeth among one another (ratio: 1.6/1.0/0.6). The positioning is performed by click, the size is then established by drawing using the finger. In particular, the image displayed on the touch screen is tapped once between the central incisors. Subsequently, the image is tapped at the height of the canine incisors. A yellow cross appears. A middle tab is displayed on the display screen. The middle tab moves the center point of the cross, this is to be placed with the horizontal directly on the cutting edge and with the vertical on the center line between the central incisors. The lateral tab, which is displayed on the display screen, is to be drawn up to the outer edge of the incisors.

During point (7) of the cosmetic dental analysis, the length-width ratio is preferably defined. This is used for judging the tooth shape. A length-width ratio of 70-80% is ideal in particular. The judgment is performed on the middle incisor, for example. The judgment can also be performed on multiple teeth successively, however. For this purpose, the selected tooth is tapped on the top left and bottom right on the display screen—a yellow rectangle appears. The rectangle is adapted to the tooth contours using the tabs which are displayed on the display screen.

During point (8) of the cosmetic analysis, the interdental triangles are preferably defined. This is performed by tapping in each case, whereby a yellow triangle appears, which can be moved using the circle to the desired point. The triangles are to become larger originating from the face center, since the contact points travel from incisal to cervical. The row of teeth thus has a looser appearance.

During point (9) of the cosmetic dental analysis, the red-white ratio is preferably defined. This is used for judging the gum curve from the central incisor to the canines (on both sides). The lateral incisor is to be shorter than the tangent between the central incisor and canine incisor. For the determination under point (9), the left side of the display screen of the touch screen is preferably tapped until a yellow line appears. By means of the circle displayed on the touch screen, the line is aligned on the zenith of the central incisor and the line is aligned on the zenith of the canine using the semicircle displayed on the touch screen. The right side of the touch screen is then tapped and the determination is also carried out as on the left side.

During point (10) of the cosmetic dental analysis, the gum papillae are preferably defined. This is used for judging the status of the gum papillae. In this case, the tooth intermediate spaces are to be well filled and no black triangles are to be visible. A yellow arrow appears by respectively tapping, which can be moved using the circle to the desired point.

1. A method for cosmetic dental analysis comprising:
a. recording an image of a face of a person via a camera,
b. transmitting the image to a computer,
c. image editing and image processing the image,
d. marking cosmetically relevant parameters on the image to create positioned markings,
e. carrying out an analysis on basis of the positioned markings via the computer,
f. displaying results of the analysis in real time on a display screen.

2. The method as claimed in claim 1,
wherein
the method is a computer-implemented method.

3. The method as claimed in claim 1,
wherein
an image region is selected after transmitting the image to the computer.

4. The method as claimed in claim 1,
wherein
initial parameters, optionally manually-input initial parameters, are communicated to the computer to characterize an image region.

5. The method as claimed in claim 4,
wherein
a unit for carrying out image editing and image processing continuously processes the manually-input initial parameters of the image region for initialization, wherein a tooth and mouth region is marked as at least one of the initial parameters.

6. The method as claimed in claim 1,
wherein
a close-up picture of a mouth region having visible upper row of teeth is selected on the display screen via said image editing and image processing.

7. The method as claimed in claim 1,
wherein
the display screen comprises a touch screen.

8. The method as claimed in claim 1,
wherein
the cosmetically relevant parameters are optionally input into the computer from at least one display screen by an input keyboard or by a mouse.

9. The method as claimed in claim 1,
wherein
the cosmetically relevant parameters are center line, laugh line, color, width of the smile, tooth axes, golden ratio, length-width ratio, interdental triangles, red-white ratio, and/or gum papillae.

10. The method as claimed in claim 1,
wherein
the camera records portrait and close-up pictures and the images are at least displayed by the display screen as a mirror image.

11. A device for a cosmetic analysis comprising:
a camera,
a computer, said camera being configured to transmit images to the computer in real time,
a unit configured to carry out image editing and image processing, wherein the device is via the computer, configured to:
analyze a tooth and mouth region of a person by marking cosmetically relevant parameters on at least one display screen via positioned markings, and
displaying results obtained in real time on the at least one display screen.

12. The device as claimed in claim 11,
wherein
the display screen comprises a touch screen.

13. The method as claimed in claim 4,
wherein the image region is a mouth and tooth region.

14. The method as claimed in claim 8,
wherein the at least one display screen is a touch screen.

15. The device as claimed in claim 12,
wherein the touch screen is configured to communicate initial parameters to the computer for characterizing an image region.

16. The device as claimed in claim 15, wherein the image region is a tooth and mouth region.
